(19) United States
(12) Patent Application Publication
(10) Pub. No.: US 2018/0256111 A1
(43) Pub. Date: Sep. 13, 2018

(54) APPARATUS AND METHOD TO RECORD HEALTH CARE VITALS AND INFORMATION ON A STAND-ALONE AND MOBILE DEVICE.

(71) Applicant: PRIMUS LLC, PALO ALTO, CA (US)

(72) Inventors: SAMEER GANAPATHY, MUMBAI (IN); SERGE SURI, MUMBAI (IN)

(21) Appl. No.: 15/659,901

(22) Filed: Jul. 26, 2017

(30) Foreign Application Priority Data
Mar. 7, 2017 (IN) 201721007870

(51) Int. Cl. A61B 5/00; A61B 5/0205; A61B 5/0432; A61B 5/15; A61B 5/157; G06F 19/00; G01N 33/49

(52) U.S. Cl. CPC A61B 5/6898; A61B 5/02055; A61B 5/0432; A61B 5/0022; A61B 5/150358; A61B 5/157; A61B 5/021; A61B 5/7264; G06F 19/3418; G06F 19/345; G06F 19/3431; G06F 19/3406; G01N 33/49; A61B 5/7445

(57) ABSTRACT

The present invention relates generally to the field of vital signs monitors, and particularly to a device and system for monitoring of the vital signs of a plurality of patients simultaneously yet independently. The health monitoring system includes a medical signal processor which communicates with a wireless or Bluetooth distributed sensor system. The health monitoring system includes the patient data collection system coupled to a communications network for transmission of patient health data from a patient data collection system to a provider analysis system.

### FIELD OF THE INVENTION

The present invention relates generally for health monitoring and more particularly to a health monitoring system that utilizes a medical signal processor with a wireless or Bluetooth distributed sensor system.

### BACKGROUND OF THE INVENTION

In General, continual vital signs monitoring requires either hands-on human attention or bulky, heavy, complicated, and expensive equipment which typically is impossible to have on hand when and where it is needed. Furthermore, in most hospitals, medical emergency situations, disaster sites, and combat zones, care for numerous patients can be difficult for a finite number of clinicians or medically trained care providers to monitor multiple patients on a continuous basis. Commercially available vital signs monitoring equipment may be helpful in these settings, however, the availability and use of such equipment in emergency situations, or in those instances where a plurality of subjects must be monitored, and decisions based on numerous casualties are made, is rarely feasible or possible with today's medical monitors. While there has been a trend to miniaturize vital signs monitoring equipment that are more effective and helpful to medical emergency personnel on site, further improvements in the effectiveness, portability, and ease of use of vital signs monitoring equipment, as well as the simplicity of wireless connectivity of the equipment is desirable, and the devices and systems for wireless monitoring of patients' vital signs of the present invention addresses the existing problems and provides related solutions and benefits.

Monitoring the health of people has always been important. As the population ages and more people advance in age health monitoring systems become more significant to maintaining a healthy lifestyle and disease management. Remote health monitoring makes it easier and cost effective to monitor the health of vast populations. Wireless systems are the most desired approach to enable remote health monitoring. Therefore, a variety of wireless health monitoring systems have been introduced over the years.

Conventional wireless health monitoring systems are bulky, expensive, have inadequate wireless link reliability and have high power dissipation which severely limits their applications, particularly to monitor wide ranging physiological parameters in high volumes for large populations. Accordingly, what is desired is a system that addresses the above-identified issues.

Recent years have seen an explosion of home based healthcare devices. Ranging from wellness products like Fitbit and Jawbone to stand alone devices for measuring Blood Pressure, Glucose Levels and ECG the market has a plethora of devices to record diagnostic data. Till date there hasn't been a single integrated healthcare diagnostic device that combines all these functionalities into one platform.

U.S. Ser. No. 12/661,757 Patent Application includes an apparatus for wireless monitoring of the vital signs of a patient, including a plurality of patient mounted vital signs monitors, each having a plurality of sensors for detecting and measuring the vital signs of a patient; a first display for displaying the patient's vital signs, the display being operatively connected to the plurality of sensors, wherein the display can receive and display the patient vital signs; a first transceiver operatively connected to the patient mounted vital signs monitor, and operable to transmit the patient's vital signs data to a remote transceiver; and a first processing means for processing the detected or measured vital signs and for controlling the operation of at least the first display and the first transceiver; a user mounted processing system and monitor including a second transceiver for wireless connection to the first transceivers of the plurality of the patient mounted vital signs monitors; a second display configured to separately display each of the patients' vital signs data received from the plurality of the patient mounted vital signs monitors; and a second processing means for processing the received vital signs data from the plurality of the patient mounted vital signs monitors and for controlling the operation of at least the second display and the second transceiver; whereby, users can monitor the vital signs of a plurality of patients by way of the patient mounted vital signs monitor or by way of the user mounted display.

U.S. Pat. No. 6,319,200 B1 patent disclose a system for monitoring physiological data representing the condition of a patient, said system comprising sensors for monitoring blood pressure, pulse rate, blood oxygen concentration and electrocardiographic information to provide the physiological data; a patient monitor coupled to the sensors for continuous, uninterrupted collection of the physiological data; a central monitoring system for storage, analysis, and display of continuous, uninterrupted physiological data collected by said patient monitor, the central monitoring system including a transmitter for transmitting a select data signal to select at least one physiological parameter to be monitored at said patient monitor; the patient monitor including a radio-frequency transmitter for transmitting said physiological data from said monitor to said central monitoring system.

U.S. Pat. No. 6,406,426 B1 patent disclose an integrated medical monitoring and alert system for monitoring a medical therapy delivered to a patient and patient physiological parameters is disclosed. The medical monitoring system preferably includes a central monitoring system and one or more of a therapeutic device, a patient monitor, and an integrated alert system. The components are linked together through a bi-directional communications system which can comprise a wireless communications link to provide for mobile patients and communications to remote caregivers.

U.S. Ser. No. 12/476,488 patent application disclose a flow measurement system includes: a respiration monitoring apparatus which measures a flow of a gas flowing through a flow path, to output a measurement value; and a gas monitoring apparatus which samples the gas to measure at least one of a concentration of a component of the gas and a component of the gas and to generate information on the flow of the sampled gas. The measurement value is corrected based on the information.

US 2009/0198110 A1 published patent application disclose a biological information acquisition telemetry system includes: a transmit device, attached to a part of a living body and configured to acquire a biological signal to transmit the biological signal as a radio signal; a relay device, configured to perform space diversity reception, the relay device including: a first receiver, configured to wirelessly receive the radio signal from the transmit device; a first transmitter, configured to transmit the radio signal received by the first receiver; and an attachment unit, adapted to hold the first receiver and the first transmitter and attach the first receiver and the first transmitter to the living body; and a center apparatus, including: a second receiver, configured to receive the radio signal from the first transmitter of the relay device; a processor, configured to generate biological information based on the radio signal received by the second receiver; and a display, configured to display the biological information generated by the processor.

Typically, continual vital signs monitoring requires either hands-on human attention or bulky, heavy, complicated, and expensive equipment which typically is impossible to have on hand when and where it is needed. Furthermore, in most hospitals, medical emergency situations, disaster sites, and combat zones, care for numerous patients can be difficult for a finite number of clinicians or medically trained care providers to monitor multiple patients on a continuous basis. Commercially available vital signs monitoring equipment may be helpful in these settings, however, the availability and use of such equipment in emergency situations is rarely feasible or possible with today's medical monitors. While there has been a trend to miniaturize vital signs monitoring equipment that are more effective and helpful to medical emergency personnel on site, further improvements in the effectiveness, portability, and ease of use of vital signs monitoring equipment, as well as the simplicity of wireless connectivity of the equipment is desirable, and the devices and systems for wireless monitoring of patients' vital signs of the present invention addresses the existing problems and provides related solutions and benefits

Clinically, it can be challenging to differentiate certain systemic viral and bacterial infections. Bacterial cultures are usually performed in cases of severe infection such as pneumonia, or when the consequence of missing a diagnosis can lead to severe complications, such as with Strep throat. Often times, cultures are difficult to obtain. Unfortunately, viral cultures are not routinely performed due to the significant time delay in receiving results. New viral screening PCR panels are useful but they are expensive and do not provide information at the point of care. Thus, there remains a need for a simple, easy to use diagnostic test that is capable of differentiating viral and bacterial infections.

Recent years have seen an explosion of home based healthcare devices. Ranging from wellness products like Fitbit and Jawbone to stand alone devices for measuring Blood Pressure, Glucose Levels and ECG the market has a plethora of devices to record diagnostic data. Till date there hasn't been a single integrated healthcare diagnostic device that combines all these functionalities into one platform.

Moreover, testing of infectious diseases and cardiac profiling has been the prerogative of massive lab based tests. In-home testing, analysis and results for such advanced tests haven't been provided for before.

Present invention not only combines all health and wellness functionalities but also has an integrated software and cloud platform to act as a permanent health assistant.

### OBJECTIVE OF THE INVENTION

Objective of the present invention is to provide a novel health monitoring system that not only combines all health and wellness functionalities but also has an integrated software and cloud platform to act as your permanent health assistant.

Yet another object of the present invention is to provide methods and compositions for diagnosis of infectious disease that is easy to administer.

It is further, an object and advantage of another exemplary embodiment of the present invention to store patient information.

It is an additional object of yet another embodiment of the invention to provide a diagnostic and identification system which communicates with a patient record management system or other device.

A further objective of this platform is to have a single score namely the Health Score that acts like an accurate indictor of a consumer's health profile to administer customized health plans and benefits.

The above and other objects, advantages and features of the present invention will become more readily appreciated and understood from a consideration of the following detailed description of preferred exemplary embodiments of the present invention when taken together with the accompanying drawings of the present invention.

### SUMMARY OF THE INVENTION

The present invention provides a wireless or Bluetooth medical signal processing system for health monitoring. The health monitoring system includes a medical signal processor which communicates with a wireless distributed sensor system as its peripheral for detecting physiological parameters of the person and for providing signals indicative thereof.

The present invention provides a lateral flow assay that is capable of detecting and differentiating viral and bacterial infections. A combined point of care diagnostic device tests markers for viral infection and markers for bacterial infection, to effectively assist in the rapid differentiation of viral and bacterial infections.

In accordance with the foregoing objects, the present invention the health monitoring system includes
a. Custom Processor(203) that allows for real-time recording, analysis and transmission of health care sensor data;
b. Blood Pressure(404), ECG(406), Temperature(403), Blood Glucose(402), SPO2(407) and other health care sensors that communicate with the processor;
c. A micro-fluidics based platform(206) for taking small blood samples;
d. An imaging array(206) for recording blood sample data for infectious disease and cardiac profiling;
e. In-built storage that keeps a fixed amount of historical diagnostic data;
f wireless or Bluetooth on-board chip(303) to transmit healthcare data to a mobile application or cloud platform;
g. mobile application that saves all healthcare data and analysis it in real-time to bring out Health Score—a single metric to keep track of your well-being;
h. The mobile application also communicates with the cloud infrastructure to enable an AI based platform for recommendations on the patients' health and well-being
i. The mobile application also allows you to consult offline or online with doctors and pharmacy vendors for delivery of medicines

### BRIEF DESCRIPTION OF THE DRAWING

The foregoing and other features and advantages of the present invention will be more fully understood from the following detailed description of illustrative embodiments, taken in conjunction with the accompanying drawings in which:

FIG. 1A is an isometric view of the health monitoring system;

FIG. 1B is an isometric view of the health monitoring system;

FIG. 1C is a top view of the health monitoring system;

FIG. 1D is a left view of the health monitoring system;

FIG. 1F is a right view of the health monitoring system;

FIG. 1E is a front view of the health monitoring system;

FIG. 1G is a back view of the health monitoring system;

FIG. 1H is a bottom view of the health monitoring system.

The health monitoring system consists of varied medical sensors such as Blood Pressure, ECG, SPO2, Blood Glucose, Temperature and others. These sensors communicate with an on-board processor and the data gets transmitted over wireless or Bluetooth to the mobile application that further communicates data to the cloud platform described in further figures.

FIG. 2: is the cross sectional view of the health monitoring system wherein,

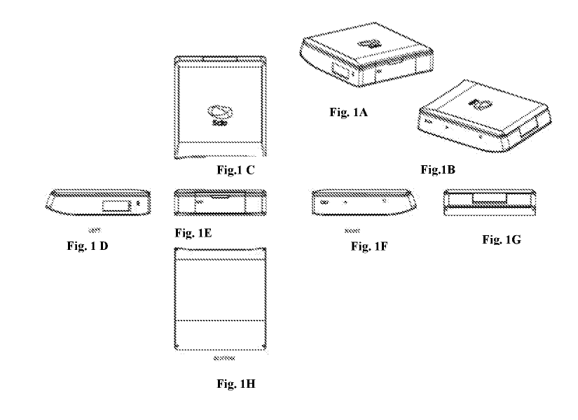

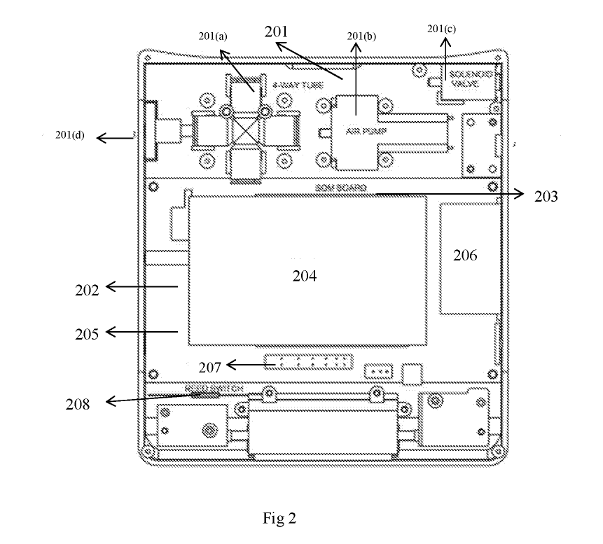

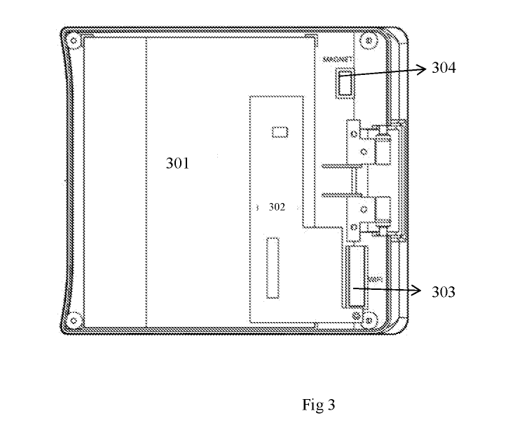

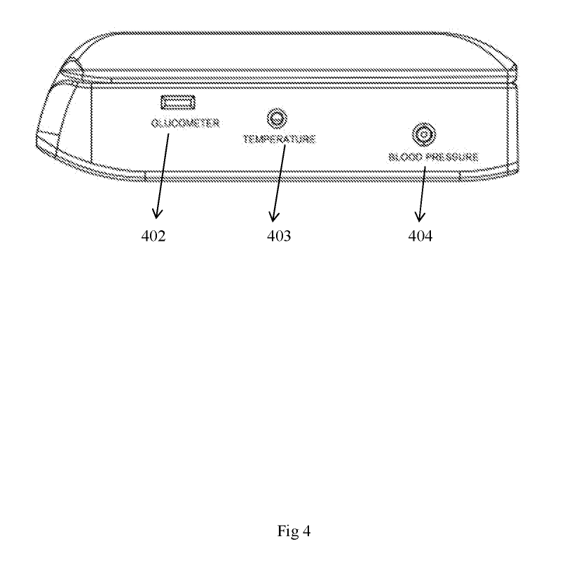

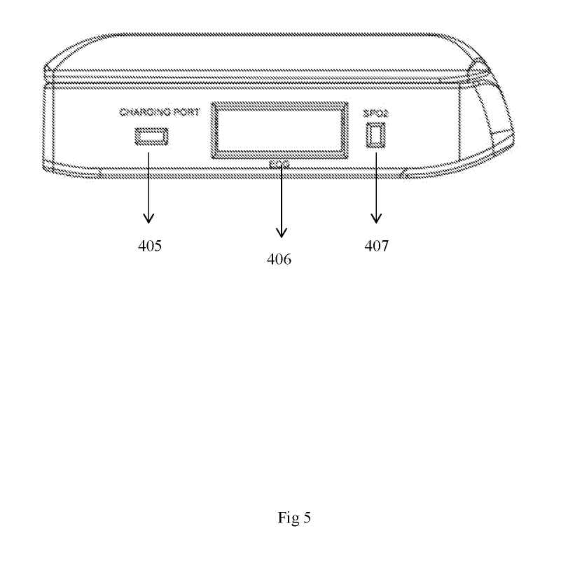

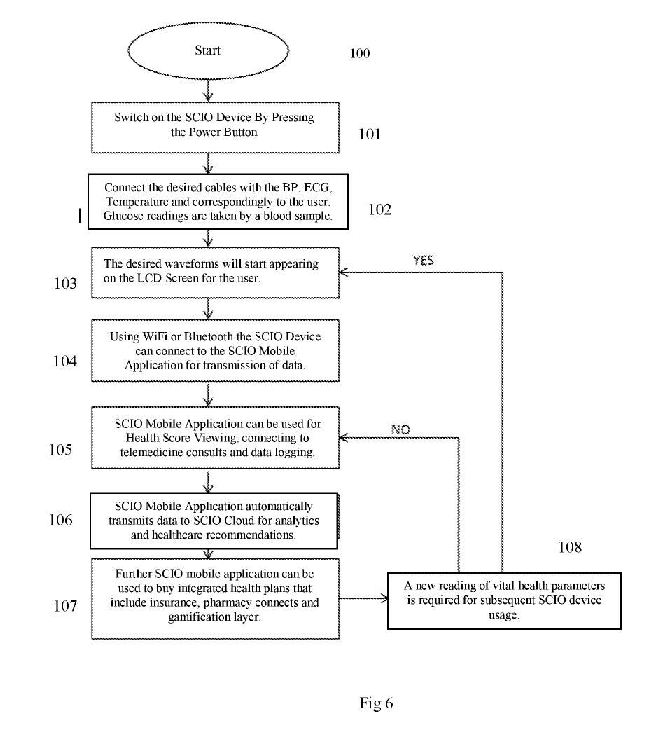

The upper part is the Blood Pressure Measurement Console(201), including 4-way Tube{201(a)}, Air-Pump{201(b)} and Solenoid Valve{201(c)}, and Plastic Connector{201(d)}. Plastic connectors are used to entangle the Blood Pressure measurement area—that helps air to pass through the 4-way pump and solenoid for accurate BP measurements.

The mid part comprise of PCB Board(202) consisting of processor (SoM)(203) Battery(204), and RAM(205). Processor (SoM)(203) is System on Module Based Processor System lies beneath Battery(204). Micro-fluidics based system along with embedded image array for recording data(206).

The lower part comprise of LCD Connector(207) for transmitting image data from sensors to LCD(301) and Reed Switch(208) detects when the device mainly the LCD(301), is closed or open.

FIG. 3: shows Liquid Crystal Display(301) combined with LCD Driver Circuit(302) to display WaveForms, WiFi+Bluetooth Module(303) for transmission of health data to cloud or mobile application and Magnet(304) is used along with reed switch to detect the opening and closing of the device.

FIG. 4: shows the right view(F) of Metal Enclosure For Component Housing(401) which comprise of ports of Glucometer Connector(402) that takes in a blood sample, Temperature Lead Connector(403) and Blood Pressure Lead Connector(404) along with arm-pad.

FIG. 5: shows the right view(D) of Metal Enclosure For Component Housing(401) which comprise of ports for charging(405), Single or 12-LED ECG Connector(406) for cardio waveforms and Finger-Touch Based SPO2 Connector(407).

FIG. 6: shows the flow diagram of the working of the health monitoring system.

### DETAILED DESCRIPTION OF THE INVENTION

It is to be understood that both the foregoing general description and the following detailed description are exemplary and explanatory only, and should not be considered restrictive of the scope of the invention, as described and claimed. Further, features and/or variations may be provided in addition to those set forth herein. For example, embodiments of the invention may be directed to various combinations and sub-combinations of the features described in the detailed description and include systems and methods for transmission of patient heart beat data from a patient work station to a remote health provider analysis system.

In an embodiment the health monitoring system of the present invention includes a metal enclosure(401) that houses the medical device sensors, processor, printed circuit boards(203), LCD screen(301) and other elements. The health monitoring system of the present invention includes On-board health sensors such Blood Pressure(404), Temperature(403), SPO2(407), ECG(406), Blood Glucose(402), to record health vitals.

In an embodiment the health monitoring system of the present invention includes On board LCD Screen(301), to display health information that can be interacted on by the patient. The PCB Board(202) consists of a micro-processor, RAM(205), Memory, Battery(204) and other passive components. Separate cables to connect each of the health sensors to the patient for recording data.

In an embodiment the health monitoring system of the present invention includes a custom micro-fluidics platform(206) that can input small blood samples and treat them chemically for further analysis and a camera based system for sensing the image and analyzing LFIs for infectious disease and cardiac panel profiling.

In preferred embodiments of the health monitoring system mobile application(104) provides a detailed interface to the health monitoring system. The mobile application allows determining Health Score based on various factors such as historical data, current health vitals and much more (105).

In preferred embodiments of the health monitoring system has in-built algorithms for health scoring: a hybrid of on-phone and cloud AI algorithms that help computes a single metric for a patient's health data. The connectivity software package allows a direct line to the cloud where all data is synced and stored. The data is further used for processing of the health score (106).

In preferred embodiments of the present invention the mobile application has various portals for Pharmacy Vendors, Doctors and Insurance companies using which the patient can make an informed decision on how to optimize his/her health (107).

In one of the embodiments of the present invention an in-built mapping, tele-medicine and ordering features are available. The application consists of in-build location based maps for Pharmacy, Doctors and Insurance vendors. It also contains software for connecting to the require service providers. It also has an in-built e-commerce portal for home delivery of various health services.

In a preferred embodiment of the present invention the extended eco-system consists of a Doctors App, Insurance App and Pharmacy App for the various service providers in the network.

In other exemplary embodiments of the present invention the health monitoring system interacts with the mobile. The health monitoring system receives input in view of the connectors that go from the device to the patient's body. The patient can see the output through a Graphical User Interface on the LCD Touchscreen. All device interactions happen through the touchscreen, GUI and the connector interface.

In a preferred embodiment The user can interact with the screen through the gestures selected from Swipe: This gesture can be used to scroll, go to the next screen or other functionalities; Pinch: This gesture can be used to zoom in and out of the health care data resolution and Tap: The tap gesture can be used to enable a functionality or detail out more features of a particular application.

The mobile interaction is based on standard mobile application as available in Android or iOS devices.

For Monitoring of the Vital Signs of a Patient, power on the health monitoring system and wait for the sensors to calibrate and boot up. Connect the required connectors for various health sensors. The health monitoring system can be used as a stand-alone for measuring your vitals along with testing for infectious diseases and cardiac panel profiling. Power on the mobile application and use the wireless or Bluetooth to communicate with the health monitoring system for setting up profile(101). The app is for transmission of health data on mobile device.

The mobile application can subsequently connect to the cloud for transmission of data to enable the AI system to kick-in and give health care recommendations(103). The mobile application can also connect to pharmacy vendors, doctors/healthcare professionals and insurance agents for further recommendations.

The Health Score is computed based on health, pathological and patient data to allow for a quantitative metric on the current state of patients health. The Health Score is further used to create personalized health plans including but not limited to health insurance services, tele-medicine and in-person consults and medicine deliveries.

Furthermore the health monitoring system is to be used by primary 4 types of customers:
a. Consumers (Patients)
b. Healthcare Professionals
c. Doctors
d. Hospitals

In either of these cases, the health monitoring system is to be used in conjunction with the mobile application as discussed in the previous sections.

Other embodiments, uses and advantages of the present invention will be apparent to those skilled in the art from consideration of the specification and practice of the invention disclosed herein.

While the foregoing description includes many details and specificities, it is to be understood that these have been included for purposes of explanation only, and are not to be interpreted as limitations of the present invention. Many modifications to the embodiments described above can be made without departing from the spirit and scope of the invention, as is intended to be encompassed by the following claims and their legal equivalents.

We claim:

1. An apparatus to record health care vitals and information comprising:
a processor(203) that allows for real-time recording, analysis and transmission of health care sensor data;
a plurality of sensors for detecting and measuring the vital signs of a patient and communicate with the processor;
a micro-fluidics based platform(206) for taking small blood samples;
an imaging array(206) for recording blood sample data for infectious disease and cardiac profiling;
a system for storage of fixed amount of historical diagnostic data;
a wireless or Bluetooth on-board chip(303) to transmit healthcare data to a mobile application or cloud;
a central monitoring system for analysis of all healthcare data in real-time to bring out and display Health Score.

2. The apparatus as claimed in claim 1 comprising:
a plurality of cables to connect each of the health sensors to the patient for recording data;
a PCB Board(202) consists of a micro-processor, RAM(205), memory and battery(204);
an on board LCD Screen(301) to display health information interacted on by the patient.

3. The apparatus as claimed in claim 1, wherein the plurality of sensors for Blood Pressure(404), Temperature(403), SPO2(407), ECG(406), Blood Glucose(402) and electrocardiographic information are provide for healthcare data.

4. The apparatus as claimed in claim 1, wherein the micro-fluidicsplatform(206) treat small blood samples chemically for analysis of infectious disease and cardiac panel profiling.

5. The apparatus as claimed in claim 1, wherein the imaging array(206) is a camera based system for sensing the image and analyzing LFIs for infectious disease and cardiac panel profiling.

6. The apparatus as claimed in claim 1, wherein the central monitoring system has in-built algorithms for health scoring comprising;
a hybrid of on-phone and cloud AI algorithms to compute a single metric for a patient's health data;
a connectivity software package to direct line to the cloud to sync and store data for processing of the health score.

7. The apparatus as claimed in claim 1 comprising; a metal enclosure(401) that houses plurality of sensors, processor, PCB Board(202), LCD screen(301) and the central monitoring system and wireless or Bluetooth on-board chip(303).

8. A method for monitoring of the vital signs of a patient record health care vitals and information comprising:
coupling a patient to the cables to connect each of the health sensor to monitor at least one of the physiological parameters wherein the apparatus is used as a stand-alone for measuring vitals along with testing for infectious diseases and cardiac panel profiling;
transmitting a select health data signal from a central monitoring system through wireless or Bluetooth for setting up profile on a mobile device;
computing the Health Score based on health, pathological parameters and patient data to allow for a quantitative metric on the current state of patients health;
displaying at least one of the said physiological parameter through graphical user interface on the LCD Touchscreen.

9. The method for monitoring of the vital signs of a patient as claimed in claim 7, wherein the central monitoring system connect to the cloud for transmission of data to AI system to give health care recommendations.

10. The method for monitoring of the vital signs of a patient as claimed in claim 7, wherein the central monitoring system connect to pharmacy vendors, doctors, healthcare professionals and insurance agents for recommendations.
